(19) United States
(12) Patent Application Publication
(10) Pub. No.: US 2016/0073971 A1
(43) Pub. Date: Mar. 17, 2016

(54) INFORMATION MANAGEMENT APPARATUS

(71) Applicant: Sharp Kabushiki Kaisha, Osaka-shi, Osaka (JP)

(72) Inventor: Takashi HIBINO, Osaka-shi (JP)

(21) Appl. No.: 14/784,969

(22) PCT Filed: Apr. 16, 2014
(86) PCT No.: PCT/JP2014/060789

§ 371 Date: Oct. 16, 2015

(30) Foreign Application Priority Data
Apr. 18, 2013 (JP) 2013-087808

(51) Int. Cl. A61B 5/00; A61B 5/0205

(52) U.S. Cl. CPC A61B 5/7425; A61B 5/02055; A61B 5/7282; A61B 5/0024

(57) ABSTRACT

An information management apparatus including: a data reception unit (11) that receives measurement information from devices (4); and a processing unit that generates, from received measurement information in at least two different formats, notification data using a common parameter in the devices (4) and notifies a display unit (20).

### TECHNICAL FIELD

The present invention relates to an information management apparatus that displays measurement information received from a plurality of measurement devices.

### BACKGROUND ART

These days, there is increasing interest in health issues such as health care for preventing diseases including lifestyle-related diseases. Accordingly, attention is being paid to management of individual health by recording measured biological information such as our body weight, body temperature, blood pressure, and blood glucose level, or health information such as calorie intake.

Conventionally, the above described biological information or health information is recorded by writing into a notebook by hand after measuring with use of a dedicated measurement device. However, since the biological information or the health information is often given as a numerical value, when writing by hand, an error is easily caused in input of the measurement result due to confusion between numerical values to be recorded. Moreover, an operation of keeping records is troublesome due to, for example, necessity of preparing a notebook, in which records have been kept till then, on hand at the time of measurement. Accordingly, it has been difficult to keep records continuously.

Thus, with recent development of electronic devices, it has become common to use a personal computer or a mobile phone as an information management apparatus and save a measurement result in the apparatus instead of recording the measurement result by handwriting. One example of such a method is that a communication function is included in a measurement device for measuring biological information, health information, or the like, and the measurement device automatically transmits a measurement result to an information management apparatus. As a scheme for transmitting data of measurement by the measurement device to the information management apparatus, there are wired communication such as a USB, and radio communication such as Bluetooth (registered trademark), Zigbee (registered trademark) and NFC (Near Field Communication). A user manages his/her daily health by using the measurement device having such a communication function and an application with which various services such as display of a graph of a measurement result and providing of advice based on the measurement result are available.

Intended use that measurement information is transmitted to one information management apparatus from a plurality of measurement devices having different communication schemes and saved is naturally expected. This is because the user tries to measure various health information and biological information by using various measurement devices. However, when a communication scheme mounted in a certain measurement device and a communication scheme mounted in another measurement device are different from each other, the information management apparatus needs to support the different communication schemes.

Generally, when communication schemes are different, procedure for exchanging data (protocols), data to be handled (types of data), and arrangement order of data (formats) also become different. Moreover, when devices in which a communication scheme is mounted are different, information of each of the devices themselves becomes different. It is difficult to implement an application which supports a plurality of different communication schemes in common. In other words, it is necessary to individually implement different applications which independently support each of the communication schemes. This problem will be described by using FIG. 15.

FIG. 15 is a view illustrating a relation between communication devices 204 and applications 210 of an information management apparatus 200 in a conventional technique. As illustrated in FIG. 15, the applications 210 which support communication schemes of the respective communication devices 204 need to be implemented. That is, a plurality of applications 210 need to be implemented. In this system, each time one new communication scheme which is able to be supported by the information management apparatus 200 is added, one new application 210 needs to be implemented additionally, which is very burdensome for a developer. Further, it is also inconvenient for a user who uses the applications 210.

Thus, for solving such a problem, PTL 1 discloses a network management method and an apparatus which allow easy function expansion.

FIG. 16 is a view illustrating a configuration of a domain network manager 300 having a network management function unit 320 which allows easy function expansion in a conventional technique. As illustrated in FIG. 16, the network management function unit 320 of the domain network manager 300 receives a management and a control request with respect to each of terminals 304 connected to switches 310, from a package distribution apparatus 305 via a network 303. In this case, the management and control request is received by a common interface (hereinafter, referred to as a common IF) which does not depend on a device of each of the terminals 304. Then, the network management function unit 320 performs conversion into a unique management and control request scheme of each of the terminals 304 to manage and control each of the terminals 304. Further, unique response and notification from each of the terminals 304 with respect to the request are responded and notified by the common IF via the network 303.

Here, the domain network manager 300 and each of the terminals 304 are replaced with an information management apparatus and a measurement device, respectively. This provides a configuration in which a measurement result is received from the measurement device by a different communication scheme, which is notified as the common IF to the package distribution apparatus 305 by the network management function unit 320.

### CITATION LIST

### Patent Literature

PTL 1: Japanese Unexamined Patent Application Publication No. 2005-252628 (Published on Sep. 15, 2005)

### SUMMARY OF INVENTION

### Technical Problem

However, in the conventional technique as described above, the network management function unit needs to notify, as the common IF, also data unique to each of the terminals in the response and notification. Thus, when a new terminal is added and new unique data is created, it becomes necessary for the common IF to support also the new unique data entirely in addition to existing unique data. Accordingly, the common IF which assumes new unique data needs to be prepared in advance, resulting that a problem is caused that a design and a configuration of the common IF become complex.

The invention has been made in view of the problem described above and an object thereof is to provide an information management apparatus in which a design and a configuration of a common notification format for notifying display means (application) of measurement information in at least two different formats, which has been from measurement devices, do not need to be made complex.

### Solution to Problem

In order to solve the aforementioned problem, an information management apparatus according to the invention is an information management apparatus provided with a display unit, which includes a reception unit that receives measurement information in at least two different formats, which is transmitted from a plurality of measurement devices which have measured a subject, and a processing unit that generates, from the received measurement information, notification data by using a parameter common in the plurality of measurement devices and notifies the display unit of the notification data.

### Advantageous Effects of Invention

According to the invention, an effect is realized in that a design and a configuration of a common notification format for notifying display means (application) of measurement information in at least two different formats, which is received from measurement devices, do not need to be made complex.

### BRIEF DESCRIPTION OF DRAWINGS

FIG. 1 is a block diagram illustrating a configuration of a main part of an information management system according to Embodiment 1 of the invention.

FIG. 2 is a flowchart illustrating a flow of processing of the information management system according to Embodiment 1 of the invention.

FIG. 3(a) is one example of a format of notification data according to Embodiment 1 of the invention and FIG. 3(b) is notification data when a measurement result of a blood pressure is incorporated in the format of the notification data.

FIG. 4 is a flowchart illustrating a flow of processing of an information management system according to a modified example 1 of the invention.

FIG. 5 is a block diagram illustrating a configuration of a main part of an information management system according to Embodiment 2 of the invention.

FIG. 6 is a flowchart illustrating a flow of processing of the information management system according to Embodiment 2 of the invention.

FIG. 7 is a flowchart illustrating a flow of processing of an information management system in a modified example of the information management system according to Embodiment 2 of the invention.

FIG. 8 is a block diagram illustrating a configuration of a main part of an information management system according to Embodiment 3 of the invention.

FIG. 9 is a flowchart illustrating a flow of processing of the information management system according to Embodiment 3 of the invention.

FIG. 10 is a block diagram illustrating a configuration of a main part of an information management system according to Embodiment 4 of the invention.

FIG. 11 is a flowchart illustrating one example of a flow of processing of the information management system according to Embodiment 4 of the invention.

FIG. 12 is a flowchart illustrating another example of a flow of processing of the information management system according to Embodiment 4 of the invention.

FIG. 13 is a block diagram illustrating a configuration of a main part of an information management system according to Embodiment 5 of the invention.

FIG. 14 is a flowchart illustrating a flow of processing of the information management system according to Embodiment 5 of the invention.

FIG. 15 is a view illustrating a relation between communication devices and applications of an information management apparatus in a conventional technique.

FIG. 16 is a view illustrating a domain network manager having a network management function unit which allows easy function expansion in a conventional technique.

### DESCRIPTION OF EMBODIMENTS

### Embodiment 1

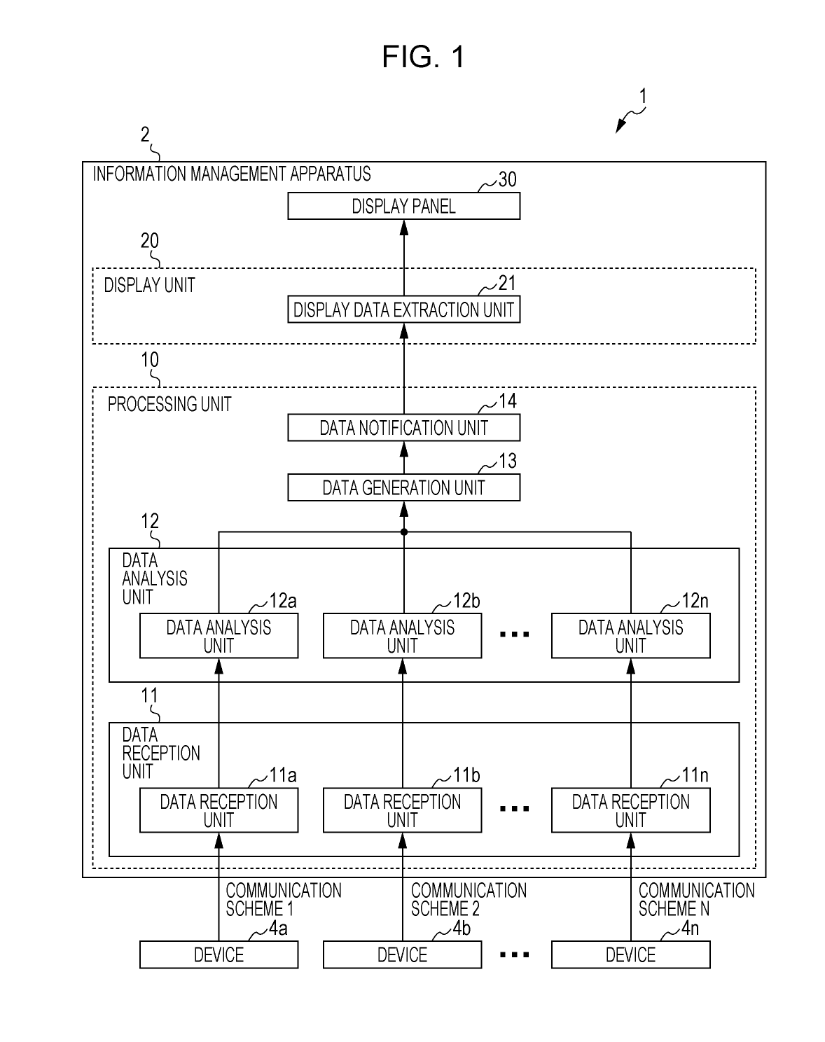

A first embodiment of the invention will be described below in detail by using FIG. 1 to FIG. 4.

### (Information Management System 1)

FIG. 1 is a block diagram illustrating a configuration of a main part of an information management system 1 according to Embodiment 1 of the invention. As illustrated in FIG. 1, the information management system 1 includes an information management apparatus 2 and a plurality of devices 4 (4a, 4b, . . . , 4n). In the present embodiment, each of the devices 4 is a measurement device which measures biological information, such as a weighing machine, a blood pressure meter and a blood glucose level meter. On the other hand, the information management apparatus 2 is an information terminal such as a personal computer, a mobile phone and a smart phone.

In the information management system 1, measurement information which is biological information about a subject measured by the device 4, is transmitted to the information management apparatus 2 by using a communication scheme mounted in each of the devices 4. The information management apparatus 2 displays the measurement information, which has been received from the device 4, on a screen regardless of a type or a communication scheme of the device 4. Examples of the communication scheme of the device 4 include a radio data communication scheme such as Bluetooth (registered trademark), NFC (Near Field Communication) or a radio LAN, and a wired data communication scheme through a USB (Universal Serial Bus) or a wired LAN.

### (Information Management Apparatus 2)

As illustrated in FIG. 1, the information management apparatus 2 is composed of a processing unit 10, a display unit 20, and a display panel 30.

The processing unit 10 receives the measurement information from the device 4, and generates notification data which is formed only of basic parameters which are common in the respective devices 4, followed by outputting to the display unit 20.

The display unit 20 acquires the notification data from the processing unit 10, and extracts information stored in the notification data to display on the display panel 30. The display unit 20 includes a display data extraction unit 21, and the display data extraction unit 21 extracts the information stored in the notification data. The display unit 20 is realized as software such as an application of a smart phone.

The display panel 30 is a display device, such as a liquid crystal display, for displaying information on a screen.

### (Processing Unit 10)

As illustrated in FIG. 1, the processing unit 10 is composed of a data reception unit 11 (reception unit), a data analysis unit 12, a data generation unit 13 (processing unit), and a data notification unit 14 (processing unit).

The data reception unit 11 receives the measurement information from the device 4. The data reception unit 11 is composed of a data reception unit 11a, a data reception unit 11b, . . . and a data reception unit 11n, which support each communication scheme of the device 4. For example, when the device 4a is a Bluetooth (registered trademark) enabled blood pressure meter and the device 4b is an NFC enabled blood pressure meter, the data reception unit 11a supports Bluetooth (registered trademark) and receives measurement information including a blood pressure value and a pulse rate from the device 4a. Similarly, the data reception unit 11b supports NFC and receives measurement information including a blood pressure value and a pulse rate from the device 4b.

The data reception unit 11 may be composed only of the data reception unit 11a that supports one communication scheme and receives measurement information in at least two different formats. For example, when being composed only of the data reception unit 11a that supports Bluetooth (registered trademark), the data reception unit 11 is able to receive data to be received from a plurality of measurement devices, as long as supporting Bluetooth (registered trademark) even if being in different formats.

The data analysis unit 12 analyzes the measurement information received by the data reception unit 11 in accordance with each communication scheme. The analysis of the measurement information is extraction of parameters of, for example, a measurement value such as a blood pressure value, unit information such as mmHg or Pa, time information indicating a time at which measurement is performed, and the like. The analysis of the measurement information is performed by an analysis processing engine mounted in each data analysis unit 12. For example, the data analysis unit 12a has an analysis processing engine which supports a Bluetooth (registered trademark) mounted in a blood pressure meter. Similarly, the data analysis unit 12b has an analysis processing engine which supports an NFC mounted in a blood pressure meter.

The data generation unit 13 stores, in a common notification format, only basic parameters which are common in the respective devices 4 among the parameters extracted by the data analysis unit 12 to thereby generate notification data. The notification format and the notification data will be described below in detail.

The data notification unit 14 outputs the notification data to the display unit 20 in order to display the notification data generated by the data generation unit 13.

### (Processing of Information Management System 1 in Embodiment 1)

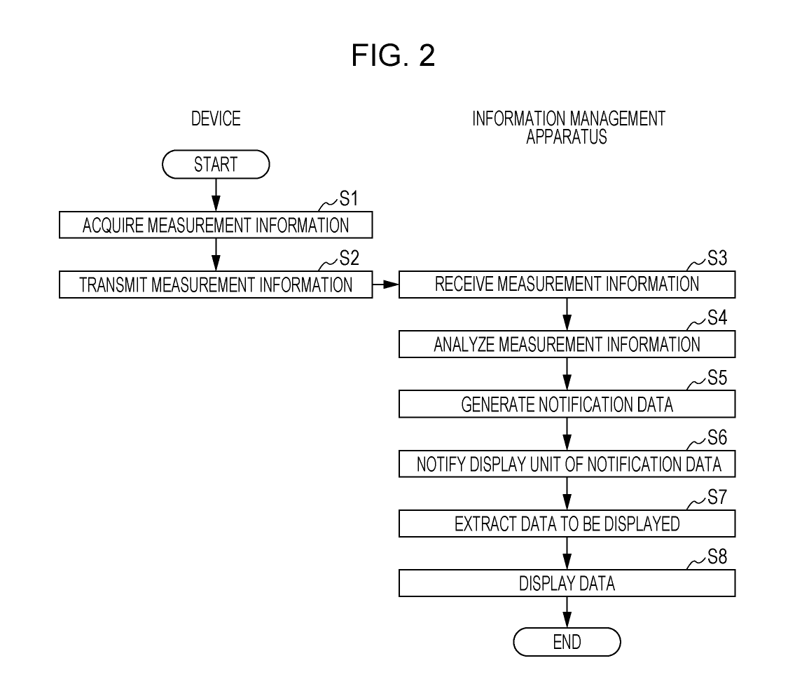

FIG. 2 is a flowchart illustrating a flow of processing of the information management system 1 according to Embodiment 1 of the invention. The flow of the processing of the information management system 1 will be described by using FIG. 2.

First, the device 4 measures a user (that is, a subject) to thereby acquire measurement information including biological information or health information of the user (step S1). The device 4 transmits the acquired measurement information to the information management apparatus 2 with a communication scheme mounted in the own device (step S2).

The data reception unit 11 of the information management apparatus 2 receives the measurement information from the device 4 (step S3). Then, the received measurement information is output to the data analysis unit 12. The data analysis unit 12 analyzes the acquired measurement information and extracts parameters such as a measurement value, unit information and time information (step S4). The extracted parameters are output to the data generation unit 13.

The data generation unit 13 stores, in a common notification format, only basic parameters which are common in the respective devices 4 among the acquired parameters to thereby generate notification data (step S5). The basic parameters are device information such as a measurement value, a measurement unit, a measurement time and a manufacturer name, and user information such as a sex and a height. The data generation unit 13 outputs the generated notification data to the data notification unit 14. The data notification unit 14 outputs the acquired notification data to the display unit 20 (step S6).

After acquiring the notification data, the display data extraction unit 21 of the display unit 20 extracts data stored in the notification data (step S7). The display unit 20 outputs the extracted data to the display panel 30, and the measurement information is displayed on the display panel 30 (step S8). The user is able to know a measurement result by viewing the display panel 30. The processing of FIG. 2 ends in this manner.

As described above, in the information management system 1, the measurement information measured by the device 4 is displayed on the display panel 30 of the information management apparatus 2.

### (Notification Format and Notification Data)

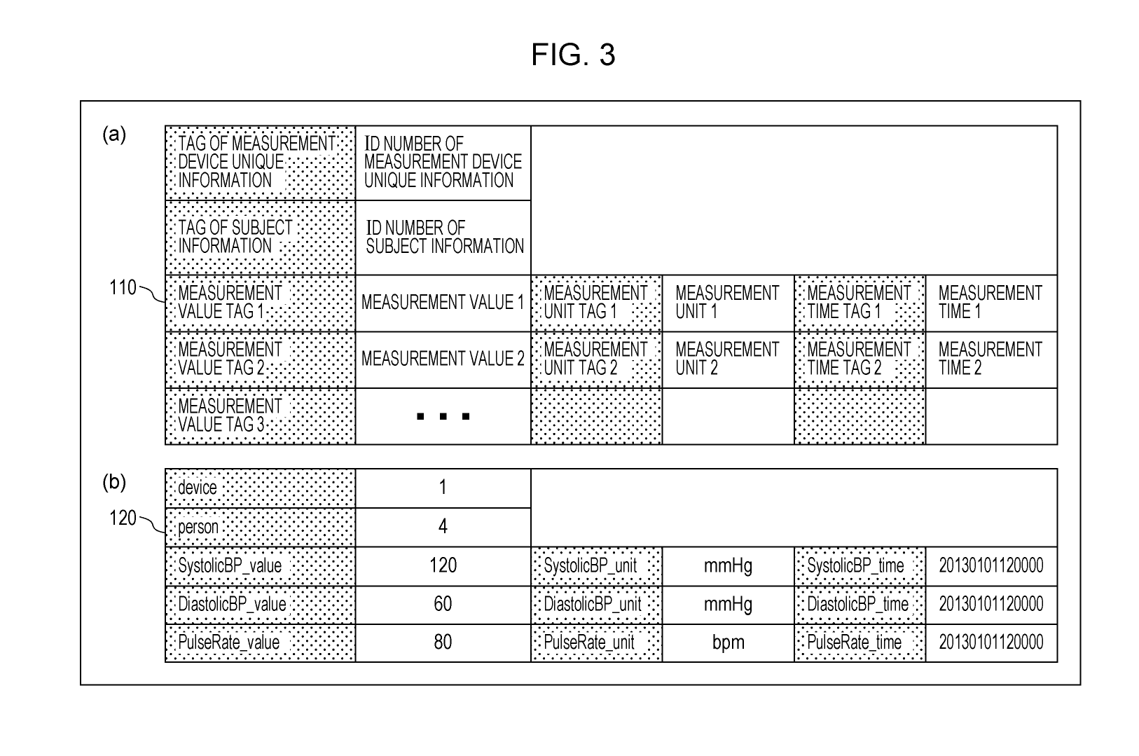

A notification format and notification data will be described by using FIG. 3. FIG. 3(a) is one example of a format 110 of notification data according to Embodiment 1 of the invention. As illustrated in the figure, the notification format 110 is formed of tag names such as a tag of measurement device unique information and a measurement value tag 1, and parameters such as an ID number of the measurement device unique information and a measurement value 1. The tag name is a character string for explaining what a corresponding parameter is. On the other hand, the parameter is a value of some sort of identifier or measurement result. The format 110 illustrated in FIG. 3(a) is common in the respective devices 4. That is, notification data using the notification format 110 illustrated in FIG. 3(a) is generated from measurement information of any device 4.

However, since the notification format 110 has the tag name abstracted, the notification format 110 is actually used after the tag name is replaced with a unique character string for each of the devices 4. As one example thereof, notification data 120 illustrated in FIG. 3(b) will be described below.

FIG. 3(b) is the notification data 120 when a measurement result of a blood pressure is stored in the format 110 used for the blood pressure. Being the measurement result of the blood pressure, each tag name of the format 110 is replaced with a character string used for the blood pressure. For example, a measurement value tag 1 and a measurement value tag 2 are changed to “SystolicBP_value” and “DiastolicBP_value”, respectively. Such notification format 110 corresponding to a blood pressure meter is prepared in advance in the information management apparatus 2.

When a measurement result of the blood pressure meter or the like is stored in the notification format 110 corresponding to the blood pressure meter, the notification data 120 illustrated in FIG. 3(b) is generated. At this time, parameters corresponding to the respective tags (such as a systolic blood pressure, a diastolic blood pressure, a mean blood pressure and a pulse rate) are stored. The parameters are not limited thereto and a unit of a measurement result (mmHg), a measurement time and the like are also stored. Furthermore, an identification number (ID number) for identifying measurement device unique information and an identification number (ID number) for identifying subject information are also able to be stored in the notification format 110.

The respective tags and the parameters corresponding thereto are all defined as having an arrangement structure in the notification format 110. Accordingly, the tags and the parameters have an arrangement structure also in the notification data 120. For example, each of an element formed of a tag representing a measurement value and the measurement value, an element formed of a tag representing a unit and the unit, and an element formed of a tag representing a measurement time and the measurement time is to be stored in the notification data 120 as one element of the arrangement.

Note that, in addition thereto, measurement device unique information and user information, which are extracted from the measurement information by the data analysis unit 12, may be also stored in the notification format 110.

Moreover, in Embodiment 1, the notification format 110 is formed only of basic parameters common in all of the devices 4, such as a measurement value, a measurement unit and a measurement time. That is, definition for storing a parameter unique to a certain specific device 4 is not made in the notification format 110. That is, such a unique parameter is not to be stored in the notification format 110.

Accordingly, in the information management system of Embodiment 1, even when the device 4 transmits a measurement result to the information management apparatus 2 by using new communication means, the existing notification format 110 is able to be used directly. Thereby, it is not necessary to vary the existing notification format 110 nor to prepare a new notification format in order to support a new communication scheme. Originally, it is not necessary to design a notification format having a complex configuration in which addition of a new communication scheme is assumed. Accordingly, with the information management system 1 of Embodiment 1, it is not required to make a design nor a configuration of a common notification format for notifying the display unit 20 (application) of measurement information received from measurement devices having different communication schemes complex.

### Modified Example 1

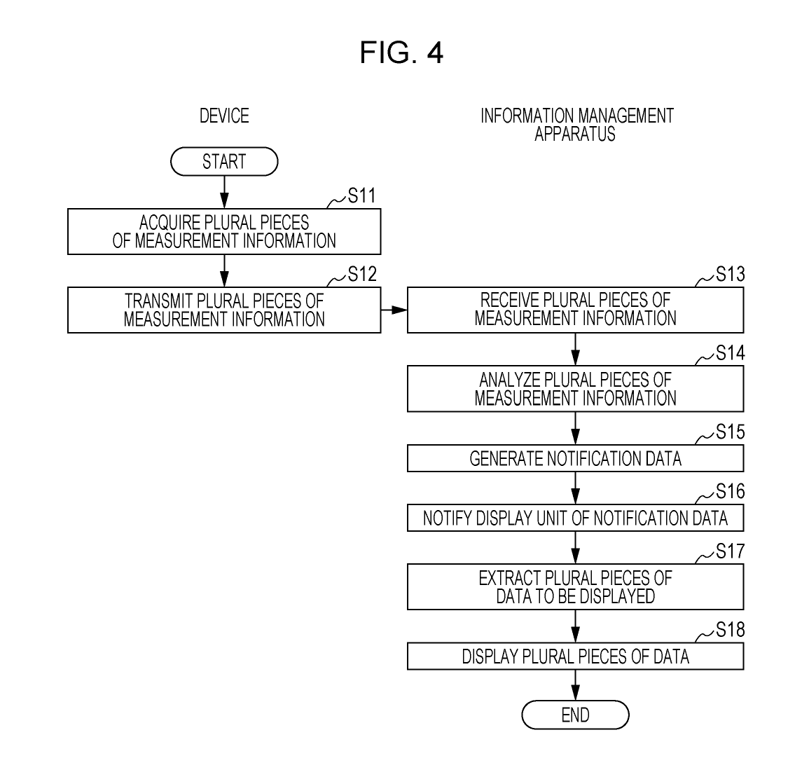

A modified example 1 obtained by modifying Embodiment 1 will be described by using FIG. 4.

FIG. 4 is a flowchart illustrating a flow of processing of an information management system 1 according to the modified example 1 of the invention.

In the present modified example, the device 4 accumulates measurement information, for example, in a case of having not transmitted the measurement information to the information management apparatus 2 or a case of having failed to transmit the measurement information. When a subject is newly measured, new measurement information and the measurement information which has been accumulated in the past are acquired (step S11). The device 4 then transmits plural pieces of acquired measurement information to the information management apparatus 2 (step S12).

The data reception unit 11 of the information management apparatus 2 receives the plural pieces of measurement information from the device 4 (step S13). Then, the plural pieces of received measurement information are output to the data analysis unit 12. The data analysis unit 12 analyzes the plural pieces of acquired measurement information, and extracts parameters such as a measurement value, unit information and time information (step S14). The data analysis unit 12 outputs the extracted parameters to the data generation unit 13.

The data generation unit 13 stores, in a notification format, only basic parameters which are common in each of the devices 4 among the acquired parameters to thereby generate notification data (step S15). At that time, since there are parameters for the plural pieces of measurement information, the parameters are stored in the notification format in a manner of repeating parameters for one measurement result. For example, when two pieces of measurement information of the blood pressure are transmitted from the same device 4 to the information management apparatus 2, first, the parameters for a first measurement result, such as a systolic blood pressure of first measurement information, a diastolic blood pressure of the first measurement information, a mean blood pressure of the first measurement information and a pulse rate of the first measurement information, are stored in the notification format. Subsequently thereto, in the same manner, the parameters for a second measurement result, such as a systolic blood pressure of second measurement information, a diastolic blood pressure of the second measurement information, a mean blood pressure of the second measurement information and a pulse rate of the second measurement information, are stored.

The generated notification data is output to the data notification unit 14. When acquiring the notification data, the data notification unit 14 outputs it to the display unit 20 (step S16).

After acquiring the notification data, the display data extraction unit 21 of the display unit 20 extracts plural pieces of data stored in the notification data (step S17). The display unit 20 outputs the extracted plural pieces of data to the display panel 30, and the measurement information is displayed on the display panel 30 (step S18). The processing of FIG. 4 ends in this manner.

Accordingly, even when the device 4 has acquired the plural pieces of measurement information, the information management apparatus 2 is able to display the plural pieces of measurement information on the display panel 30 of the information management apparatus 2.

Note that, the data generation unit 13 may generate plural pieces of notification data by storing parameters for individual measurement results in individual notification formats separately.

### Embodiment 2

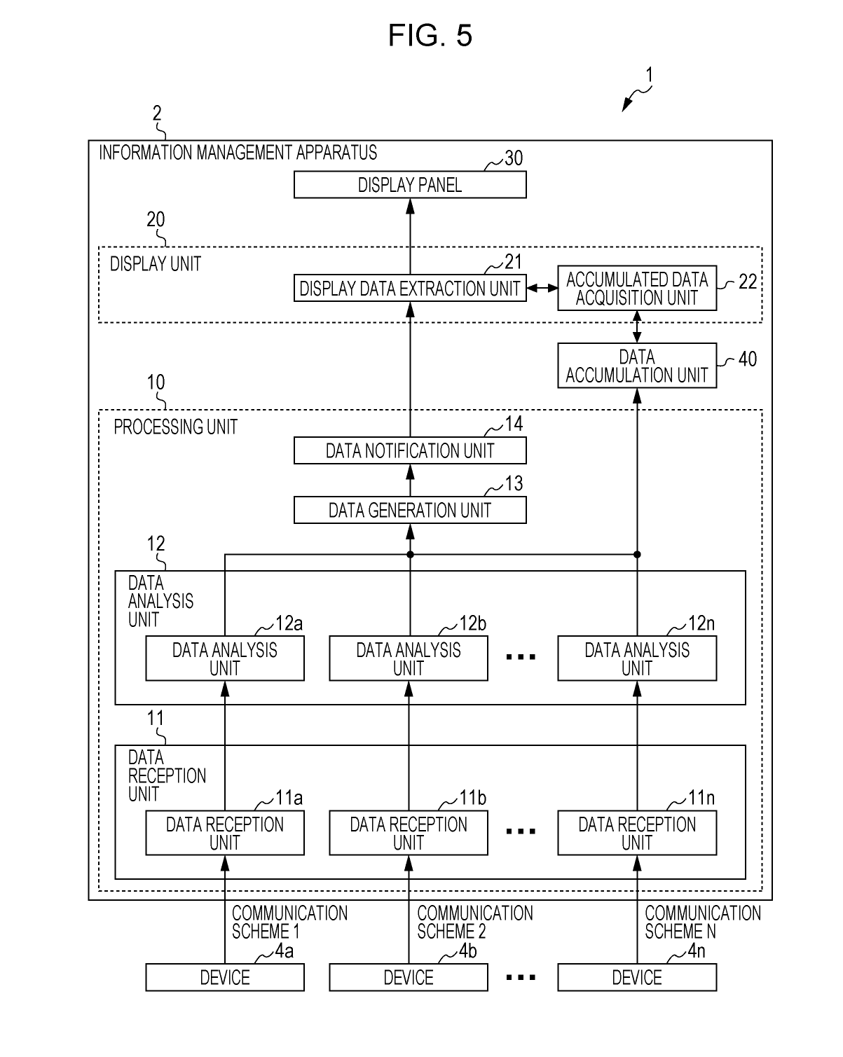

A second embodiment of the invention will be described below in detail by using FIG. 5 to FIG. 7. Note that, for convenience of description, the same reference signs are assigned to members having the same functions as those of the members described in the aforementioned embodiment, and description thereof will be omitted.

FIG. 5 is a block diagram illustrating a configuration of a main part of an information management system 1 according to Embodiment 2 of the invention. In the present embodiment, in addition to the block diagram of the information management apparatus 2 of Embodiment 1 described above, an accumulated data acquisition unit 22 of the display unit 20 and a data accumulation unit 40 (unique information recording unit, subject information recording unit) are newly included.

The accumulated data acquisition unit 22 acquires data accumulated in the data accumulation unit 40.

The data accumulation unit 40 accumulates information unique to the device 4, such as a manufacturer name and a model number of the device 4, which is extracted by the data analysis unit 12 and an ID number of the measurement device unique information for identifying the unique information, so as to be in association with each other. Note that, the ID number is allocated to the extracted unique information by the data analysis unit 12.

### (Processing of Information Management System 1 in Embodiment 2)

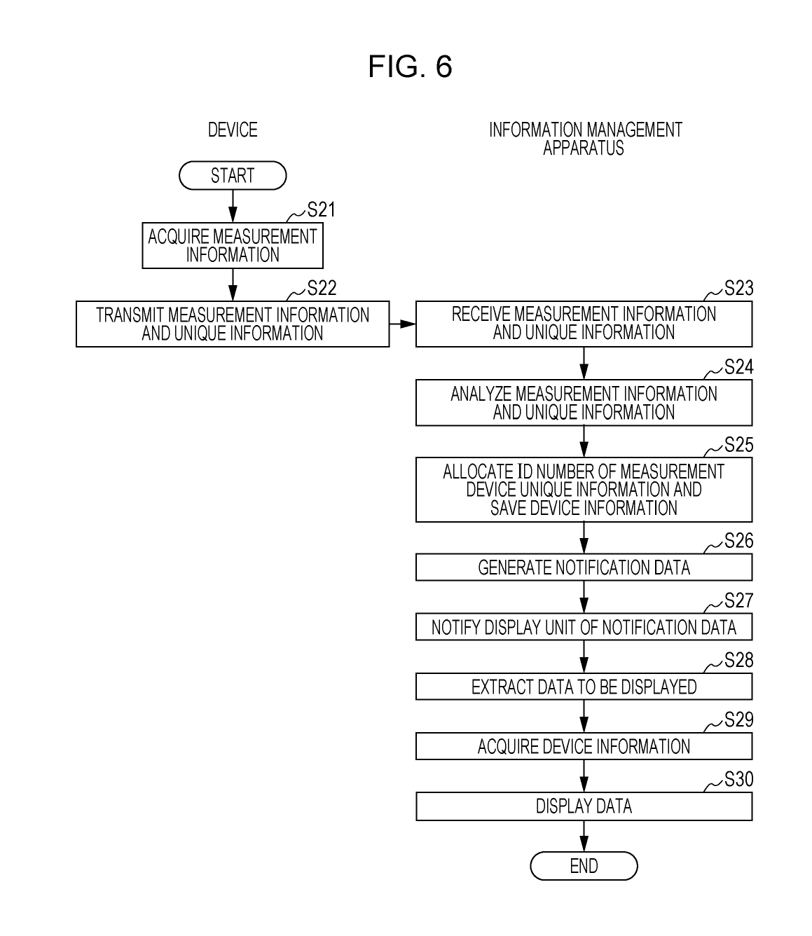

A flow of processing of the information management system 1 in Embodiment 2 will be described by using FIG. 6. FIG. 6 is a flowchart illustrating the flow of the processing of the information management system 1 according to Embodiment 2 of the invention.

First, the device 4 measures a user to thereby acquire measurement information (step S21). The device 4 then transmits the acquired measurement information and unique information which includes own device information to the information management apparatus 2 (step S22).

The data reception unit 11 of the information management apparatus 2 receives the measurement information and the unique information from the device 4 (step S23). Then, the measurement information and the unique information, which have been received, are output to the data analysis unit 12. The data analysis unit 12 analyzes the acquired measurement information, and extracts parameters such as a measurement value, unit information and time information. Similarly, the data analysis unit 12 analyzes the acquired unique information and extracts device information such as a manufacturer name and a model number (step S24).

The data analysis unit 12 allocates, to the extracted device information of the unique information, an ID number of the measurement device unique information for identifying unique information of each measurement device, and saves the extracted device information and the ID number of the measurement device unique information in the data accumulation unit 40 (step S25). Further, the parameters of the measurement information and the ID number of the measurement device unique information are output to the data generation unit 13.

The data generation unit 13 stores, in a notification format, only basic parameters which are common in each of the devices 4 among the acquired parameters. Additionally, the ID number of the measurement device unique information is also stored in the notification format. Thereby, notification data is generated (step S26). The data generation unit 13 outputs the generated notification data to the data notification unit 14. When acquiring the notification data, the data notification unit 14 outputs it to the display unit 20 (step S27).

After acquiring the notification data, the display data extraction unit 21 of the display unit 20 extracts information stored in the notification data (step S28). At this time, when the ID number of the measurement device unique information has been stored, the accumulated data acquisition unit 22 is requested to acquire device information of the ID number of the measurement device unique information, which has been stored. Since the ID number of the measurement device unique information has been allocated to the device information saved in the data accumulation unit 40 at step S25, the accumulated data acquisition unit 22 acquires the device information of the corresponding ID number of the measurement device unique information from the data accumulation unit 40 to output to the display data extraction unit 21 (step S29).

The display unit 20 outputs the extracted information and the acquired device information to the display panel 30. As a result thereof, the measurement information and the device information of the device 4 are displayed on the display panel 30 (step S30). In this manner, the processing of FIG. 6 ends.

Accordingly, in Embodiment 2, by using an element of the format 110 of the notification data of FIG. 3, which is formed of a tag of the measurement device unique information and the ID number of the measurement device unique information, the device information of the device 4, such as a manufacturer name and a model number, is able to be displayed on the display panel 30.

### Modified Example 2

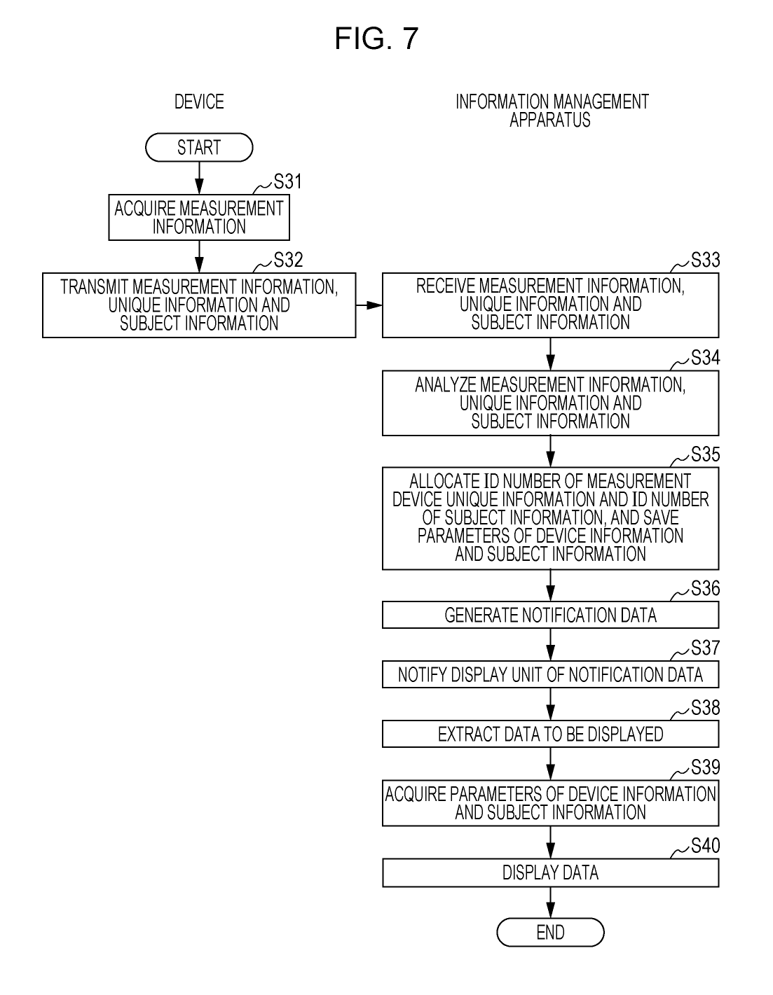

A modified example 2 obtained by modifying Embodiment 2 will be described by using FIG. 7.

FIG. 7 is a flowchart illustrating a flow of processing of an information management system 1 in the modified example 2 of the information management system according to Embodiment 2 of the invention. In the present modified example 2, in addition to measurement information and unique information, the device 4 further transmits subject information, such as a sex and a height of a subject, to the information management apparatus 2.

A user registers the subject information which is information about himself/herself in the device 4 in advance. The device 4 stores the information therein.

As illustrated in FIG. 7, first, the device 4 measures a subject to thereby acquire measurement information (step S31). The device 4 transmits the acquired measurement information, unique information which includes own device information and the subject information to the information management apparatus 2 (step S32).

The data reception unit 11 of the information management apparatus 2 receives the measurement information, the unique information and the subject information from the device 4 (step S33). Then, the measurement information, the unique information and the subject information, which have been received, are output to the data analysis unit 12. The data analysis unit 12 analyzes the acquired measurement information, and extracts parameters such as a measurement value, unit information and time information. Similarly, the data analysis unit 12 analyzes the acquired unique information and extracts device information such as a manufacturer name and a model number. Further, similarly, the data analysis unit 12 analyzes the acquired subject information and extracts parameters of a sex, a height and the like (step S34).

The data analysis unit 12 then allocates, to the extracted device information of the unique information, an ID number of the measurement device unique information for identifying unique information of each measurement device, and saves the extracted device information and the ID number of the measurement device unique information in the data accumulation unit 40. Similarly, the data analysis unit 12 allocates, to the extracted parameters of the subject information, an ID number of the subject information for identifying the subject, and saves the extracted parameters of the subject information and the ID number of the subject information in the data accumulation unit 40 (step S35). Further, the parameters of the measurement information, the ID number of the measurement device unique information, and the ID number of the subject information are output to the data generation unit 13.

The data generation unit 13 stores, in a notification format, only basic parameters which are common in each of the devices 4 among the acquired parameters of the measurement information. Additionally, the ID number of the measurement device unique information and the ID number of the subject information are also stored in the notification format. Thereby, notification data is generated (step S36). The data generation unit 13 outputs the generated notification data to the data notification unit 14. When acquiring the notification data, the data notification unit 14 notifies the display unit 20 (step S37).

After acquiring the notification data, the display data extraction unit 21 of the display unit 20 extracts information stored in the notification data (step S38). At this time, when the ID number of the measurement device unique information and the ID number of the subject information have been stored, the accumulated data acquisition unit 22 is requested to acquire device information of the ID number of the measurement device unique information and the parameters of the ID number of the subject information. The ID number of the measurement device unique information has been allocated to the device information saved in the data accumulation unit 40 and the ID number of the subject information have been allocated to the parameters of the subject information at step S35. Thus, the accumulated data acquisition unit 22 acquires the corresponding parameters of the ID number of the measurement device unique information and the corresponding parameters of the ID number of the subject information from the data accumulation unit 40 to output to the display data extraction unit 21 (step S39).

The display unit 20 outputs the extracted information, and the device information and the parameters of the subject information, which have been acquired, to the display panel 30. Thereby, a measurement result, the device information of the device 4, and the subject information are displayed on the display panel 30 (step S40). In this manner, the processing of FIG. 7 ends.

Accordingly, in the present modified example, by using an element of the notification data format 110 of FIG. 3, which is formed of a tag of the subject information and the ID number of the subject information, information of the subject, such as a sex and a height of the subject, is able to be displayed on the display panel 30.

### Embodiment 3

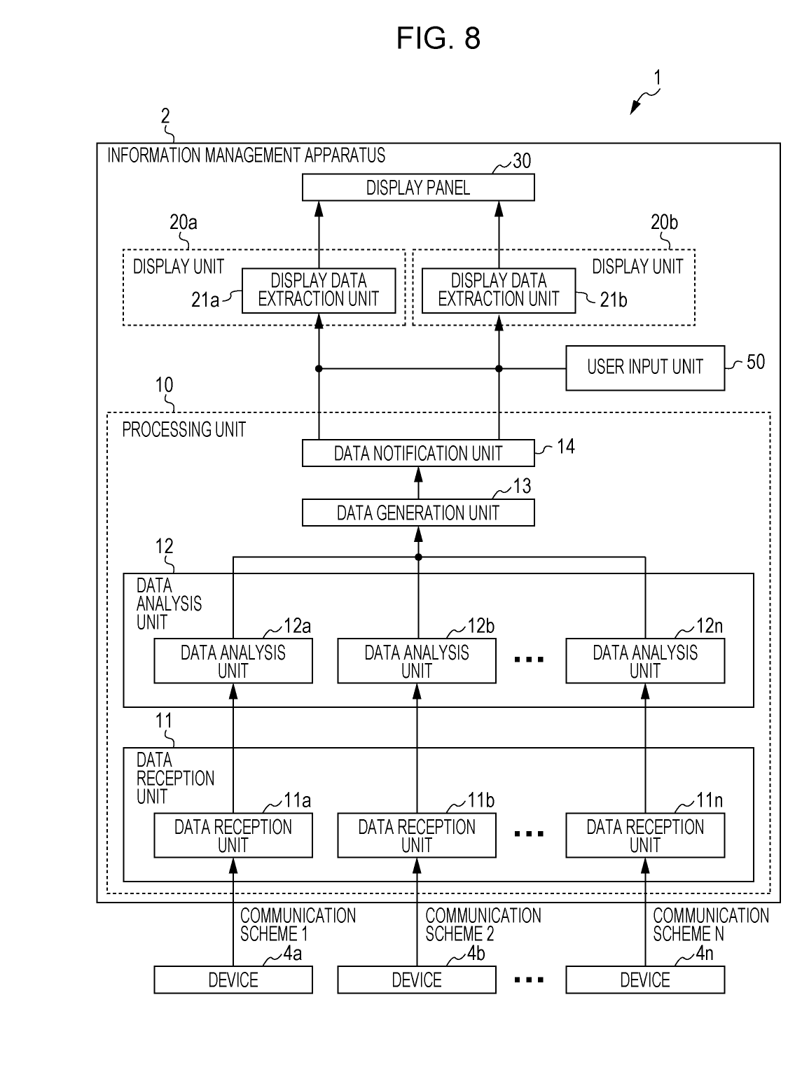

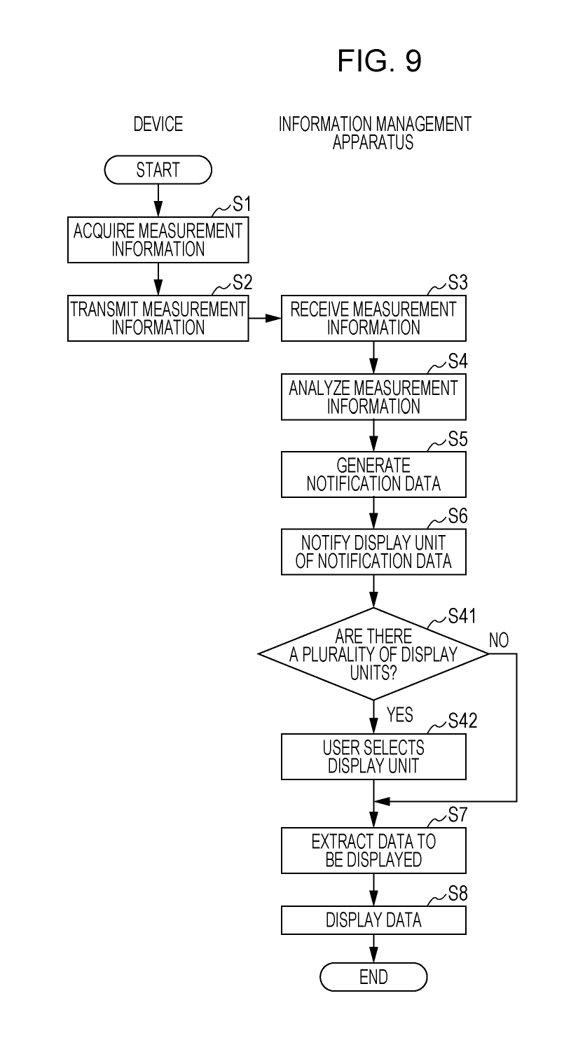

A third embodiment of the invention will be described below in detail by using FIG. 8 and FIG. 9. Note that, for convenience of description, the same reference signs are assigned to members having the same functions as those of the members described in the aforementioned embodiments, and description thereof will be omitted.

FIG. 8 is a block diagram illustrating a configuration of a main part of an information management system 1 according to Embodiment 3 of the invention. In the present embodiment, a display unit 20a and a display unit 20b are included instead of the display unit 20 of the block diagram of the information management apparatus 2 in Embodiment 1 described above, and a user input unit 50 is newly included.

As described above, the display unit 20 is realized by software such as an application of a smart phone. Accordingly, the information management apparatus 2 may include a plurality of applications, such as the display unit 20a for displaying measurement information as a graph and the display unit 20b for displaying measurement information as a text, as the display unit 20.

The user input unit 50 is hardware which receives an input of a user, such as a keyboard or a touch panel.

### (Processing of Information Management System 1 in Embodiment 3)

FIG. 9 is a flowchart illustrating a flow of processing of the information management system 1 according to Embodiment 3 of the invention. The flow of the processing of the information management system 1 in Embodiment 3 will be described by using FIG. 9. Since the procedure up to the processing for outputting the notification data to the display unit 20 by the data notification unit 14 of the information management apparatus 2 (step S6) is the same as that of the aforementioned embodiments, description thereof will be omitted.

After having notified each display unit 20 of the notification data, the data notification unit 14 decides whether or not there are a plurality of display units 20 (step S41).

When it is decided that “there are a plurality of display units 20” at step S41 (step S41: Yes), the information management apparatus 2 prompts a user to select any of the plurality of display units 20. The user selects any of the display units 20 by using the user input unit 50 (step S42). When the display unit 20 is selected by the user, only the selected display unit 20 moves to processing for extracting data (step S7), and the followings are the same as those of each of the aforementioned embodiments, so that the description thereof will be omitted.

On the other hand, when it is decided that “there are not a plurality of display units 20” at step S41 (step S41; No), the processing moves to step S7, and the followings are the same as those of the aforementioned embodiments, so that the description thereof will be omitted.

In this manner, even when the information management apparatus 2 includes the plurality of display units 20, it is possible to limit the display unit 20 by selection by the user. Further, when the user has selected the display unit 20 once, only the selected display unit 20 may automatically extract and display data.

### Modified Example 3

A modified example 3 obtained by modifying Embodiment 3 will be described.

In the present modified example, a data name of notification data includes a character string indicating a type of data. For example, a data name of notification data generated based on measurement information from a blood pressure meter includes some sort of character string indicating that the data is associated with a blood pressure, such as “blood pressure”. The character string is included in the data name when the data generation unit 13 generates the notification data.

The display unit 20 reads the data name of the notification data and decides whether or not to be display target data to be displayed by its own. When it is decided as being the display target data, the decision is returned to the data notification unit 14, and data to be displayed is extracted from the notification data and displayed on the display panel 30. When responses are given from a plurality of display units 20 to the data notification unit 14, the information management apparatus 2 may cause a user to select the display unit 20 so that only the display unit 20 selected by the user displays the data.

As described above, by storing a data name in notification data to notify the display unit 20, the display unit 20 on which data is to be displayed is automatically selected, and measurement information is displayed on the display panel 30. Accordingly, it is possible to easily prevent that data which is not to be displayed essentially is erroneously displayed, for example, such as that the display unit 20 for displaying a blood pressure displays a measurement result by a weighing machine.

### Embodiment 4

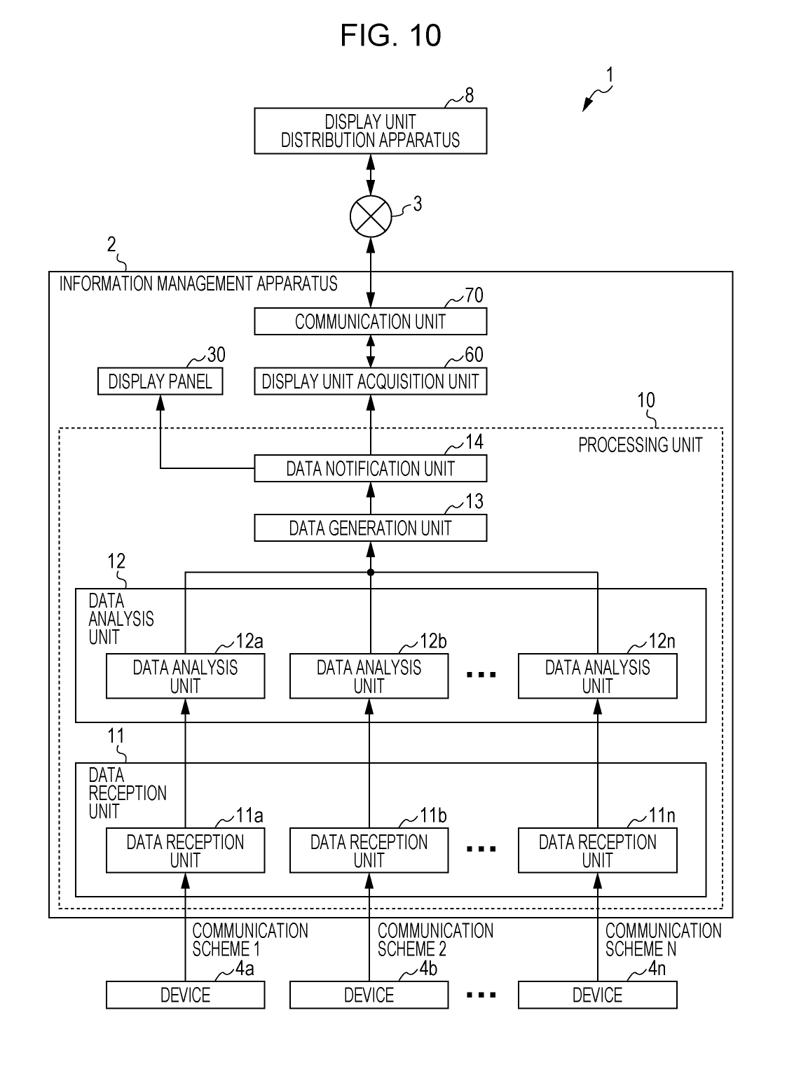

A fourth embodiment of the invention will be described below in detail by using FIG. 10 to FIG. 12. Note that, for convenience of description, the same reference signs are assigned to members having the same functions as those of the members described in the aforementioned embodiments, and description thereof will be omitted.

FIG. 10 is a block diagram illustrating a configuration of a main part of an information management system 1 according to Embodiment 4 of the invention. In the present embodiment, a network 3 and a display unit distribution apparatus 8 are newly included in the information management system 1 of Embodiment 1 described above, and a display unit acquisition unit 60 and a communication unit 70 are included instead of the display unit 20 of the information management apparatus 2.

The display unit distribution apparatus 8 is able to communicate with the information management apparatus 2 mutually through the network 3. The display unit distribution apparatus 8 is an apparatus which distributes the display unit 20 of the information management apparatus 2. As described above, the display unit 20 is software such as an application of a smart phone, and therefore is able to be distributed to the information management apparatus 2 through the network 3.

The display unit acquisition unit 60 has a function of acquiring the display unit 20 of the information management apparatus 2 from the display unit distribution apparatus 8.

The communication unit 70 is connected to the network 3 and has a function of performing bidirectional communication with the display unit distribution apparatus 8.

### (Processing of Information Management System 1 in Embodiment 4)

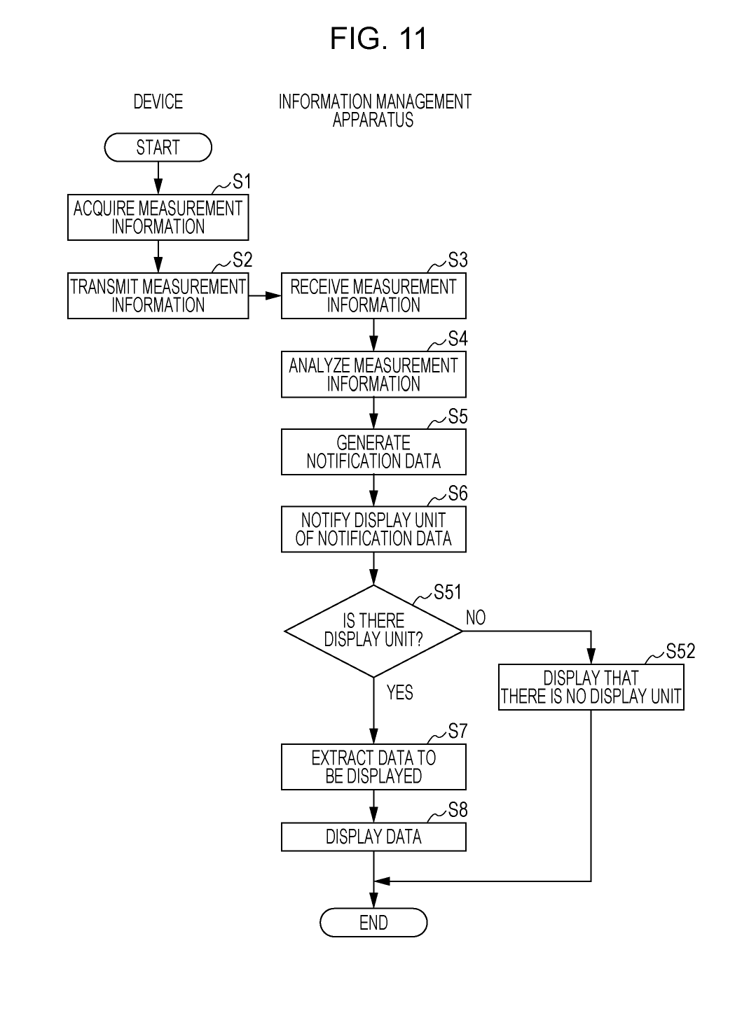

FIG. 11 is a flowchart illustrating a flow of processing of the information management system according to Embodiment 4 of the invention. The flow of the processing of the information management system 1 in Embodiment 4 will be described by using FIG. 11. Since the procedure up to the processing for outputting the notification data to the display unit 20 by the data notification unit 14 of the information management apparatus 2 (step S6) is the same as that of the aforementioned embodiments, description thereof will be omitted.

After outputting the notification data to the display unit 20, the data notification unit 14 decides whether or not there is the display unit 20 in the information management apparatus 2 (step S51).

When it is decided that “there is the display unit” at step S51 (step S51: Yes), the procedure moves to processing for extracting data by the display unit 20 (step S7), and the followings are the same as those of the aforementioned embodiments, so that the description thereof will be omitted.

On the other hand, when it is decided that “there is no display unit” at step S51 (step S51: No), the data notification unit 14 outputs a message indicating that there is no display unit 20 to the display panel 30 (step S52). Thereby, the message is displayed on the display panel 30. The processing of FIG. 11 ends in this manner.

Accordingly, even when the information management apparatus 2 does not include the display unit 20, it is possible to notify the user of it. Accordingly, the user is not required to be disturbed even when a measurement result is not displayed.

### (Automatic Acquisition of Display Unit 20)

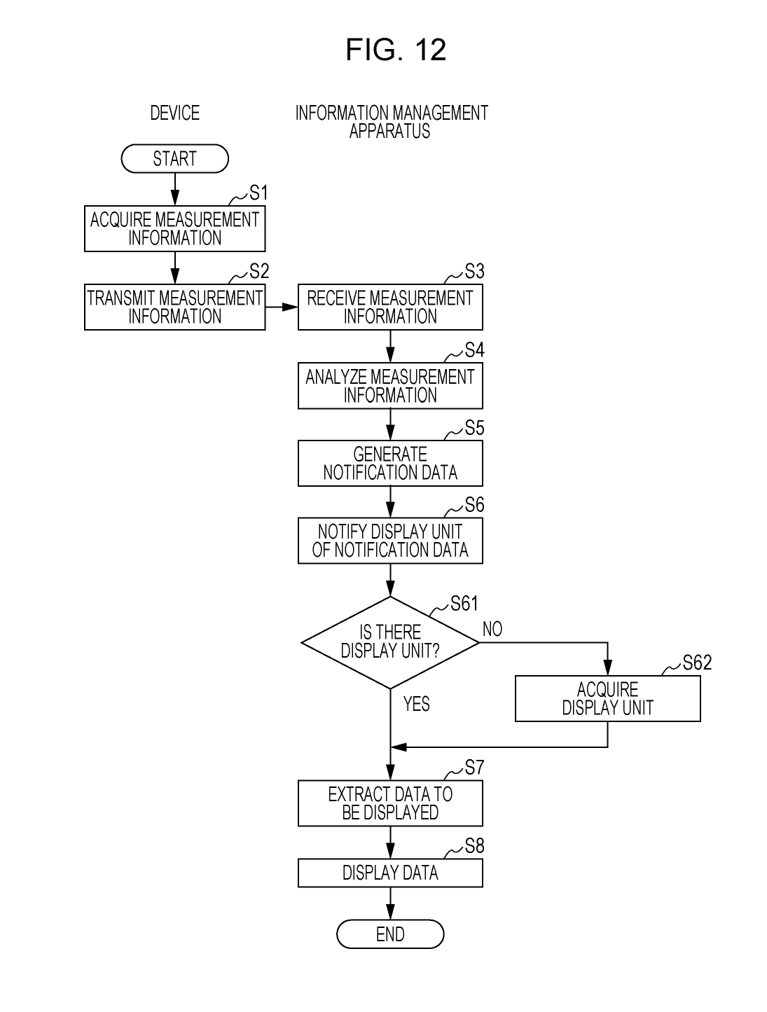

Processing for automatically acquiring the display unit 20 by using the display unit acquisition unit 60 and the communication unit 70 will be described with FIG. 12.

FIG. 12 is a flowchart illustrating another example of a flow of processing of the information management system according to Embodiment 4 of the invention. Since the procedure up to the processing for outputting the notification data to the display unit 20 by the data notification unit 14 of the information management apparatus 2 (step S6) is the same as that of the aforementioned embodiments, description thereof will be omitted.

After outputting the notification data to the display unit 20, the data notification unit 14 decides whether or not there is the display unit 20 in the information management apparatus 2 (step S61).

When it is decided that “there is the display unit” at step S61 (step S61: Yes), the display unit 20 moves to processing for extracting data (step S7), and the followings are the same as those of the aforementioned embodiments, so that the description thereof will be omitted.

On the other hand, when it is decided that “there is no display unit” at step S61 (step S61: No), the data notification unit 14 requests the display unit acquisition unit 60 to acquire the display unit 20. In response thereto, the display unit acquisition unit 60 outputs a request signal for acquiring the display unit 20 to the communication unit 70. The communication unit 70 transmits the acquired request signal to the display unit distribution apparatus 8 through the network 3.

The display unit distribution apparatus 8 receives the request signal transmitted to the network 3 and transmits the display unit 20 corresponding to the request signal to the communication unit 70 through the network 3.

The communication unit 70 receives the display unit 20 from the network 3 to output to the display unit acquisition unit 60. Thereby, the display unit acquisition unit 60 acquires the display unit 20 (step S62). The information management apparatus 2 installs the acquired display unit 20 on its own. Thereby, the information management apparatus 2 is to include the display unit 20. Thus, the data notification unit 14 notifies the display unit 20 which has been installed of the notification data. The procedure moves to the processing in which the display unit 20 extracts data (step S7), and the followings are the same as those of the aforementioned embodiments, so that the description thereof will be omitted.

In this manner, even when the information management apparatus 2 does not include the display unit 20, it is possible to automatically acquire the display unit 20. As a result thereof, a measurement result is displayed on the display panel 30 normally. Accordingly, it is possible to prevent that the user is confused because the measurement result is not displayed.

### Embodiment 5

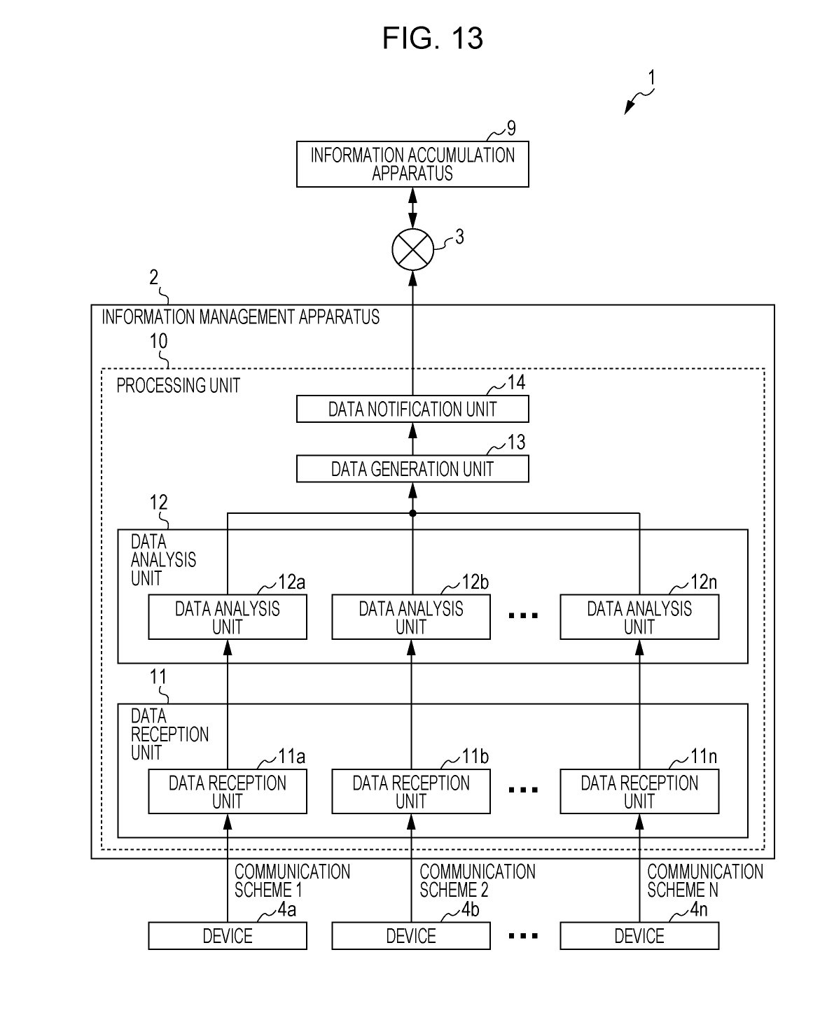

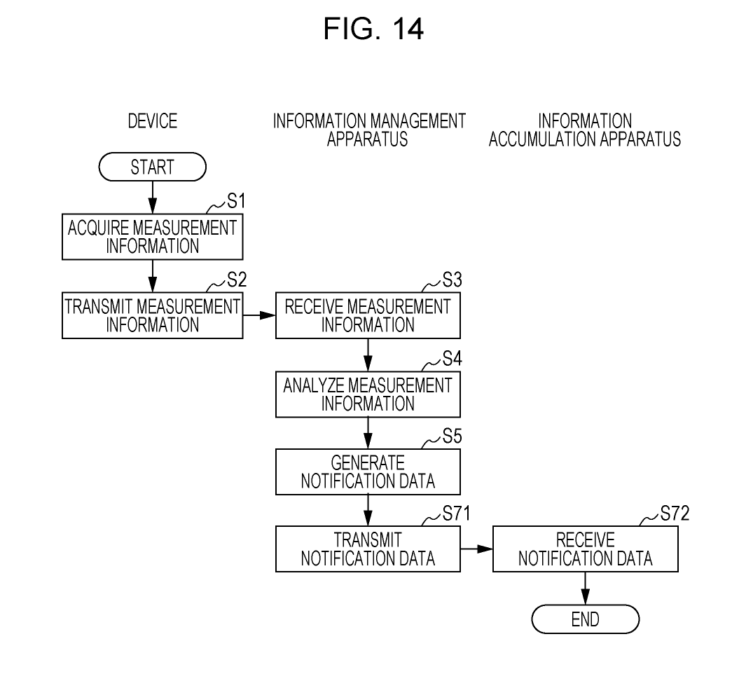

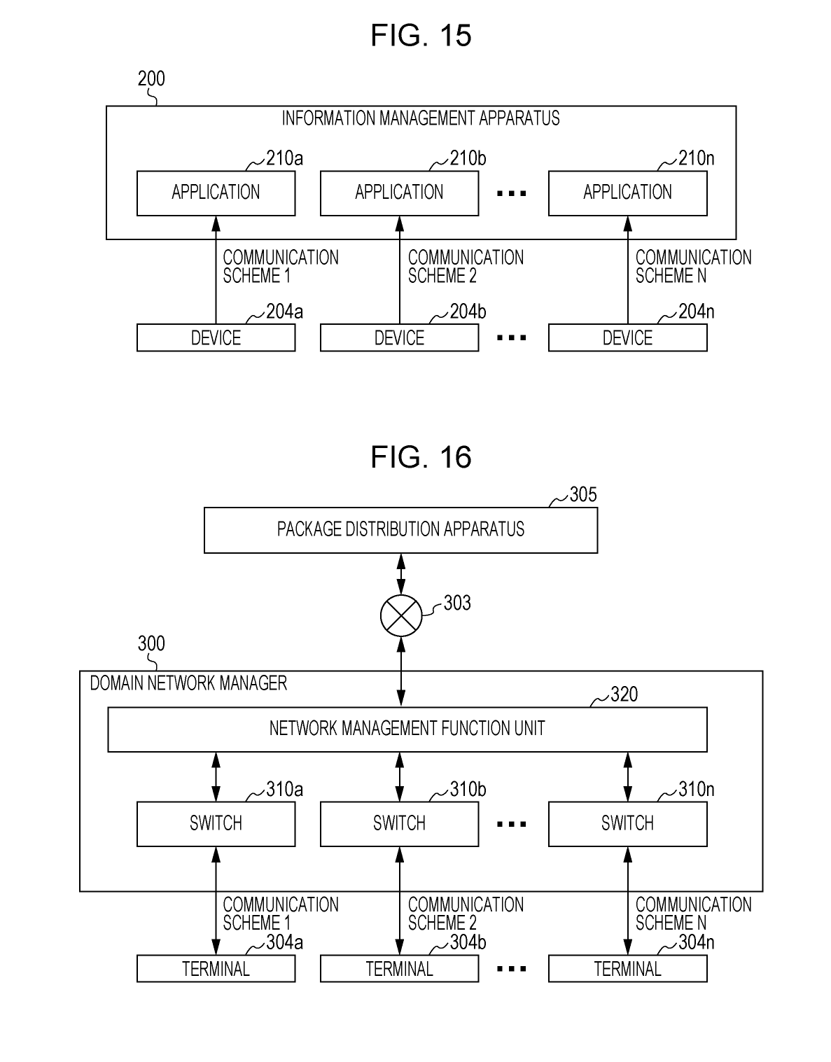

A fifth embodiment of the invention will be described below in detail by using FIG. 13 and FIG. 14. Note that, for convenience of description, the same reference signs are assigned to members having the same functions as those of the members described in the aforementioned embodiments, and description thereof will be omitted.

FIG. 13 is a block diagram illustrating a configuration of a main part of an information management system 1 according to Embodiment 5 of the invention. In the present embodiment, the network 3 and an information accumulation apparatus 9 are newly included in the information management system 1 of Embodiment 1 described above, and the information management apparatus 2 does not include the display unit 20 nor the display panel 30.

The information accumulation apparatus 9 is able to communicate with the information management apparatus 2 mutually through the network 3. The information accumulation apparatus 9 receives and accumulate notification data generated by the information management apparatus 2.

### (Processing of Information Management System 1 in Embodiment 5)

FIG. 14 is a flowchart illustrating a flow of processing of the information management system 1 according to Embodiment 5 of the invention. Since the procedure up to the processing for generating the notification data by the data generation unit 13 of the information management apparatus 2 (step S5) is the same as that of the aforementioned embodiments, description thereof will be omitted.

The data generation unit 13 outputs the generated notification data to the data notification unit 14. The data notification unit 14 transmits the acquired notification data to the information accumulation apparatus 9 through the network 3 (step S71). The information accumulation apparatus 9 receives the notification data from the network 3 (step S72) for accumulation. Note that, the data notification unit 14 may output the notification data to the display unit 20 and transmit it to the information accumulation apparatus 9 only when there is no response from the display unit 20.

In this manner, when the information management apparatus 2 and the information accumulation apparatus 9 are allowed to perform communication through the network 3, it is possible to transmit the notification data to the information accumulation apparatus 9 and accumulate measurement information. Further, the information accumulation apparatus 9 is able to receive the notification data which is made still from a common notification format regardless of the device 4, and therefore does not need to have functions corresponding to the individual devices 4.

### Example of Realization by Software

Each block of the information management apparatus 2 may be realized by a logic circuit (hardware) formed in an integrated circuit (IC chip) or the like, or may be realized by software by using a CPU (Central Processing Unit).

In the latter case, the information management apparatus 2 includes, for example, a CPU which executes a command of a program, which is software realizing each function, a ROM (Read Only Memory) or a storage apparatus (which is referred to as a “recording medium”) in which the program and various data are recorded so as to be readable by a computer (or the CPU), and a RAM (Random Access Memory) which develops the program. The computer (or CPU) reads the program from the recording medium for execution, so that the object of the invention is achieved. As the recording medium, a “non-temporal tangible medium”, for example, such as a tape, a disc, a card, a semiconductor memory, and a programmable logic circuit may be used. Further, the program may be supplied to the computer via any transmission medium (communication network, broadcast waves, etc.) which is capable of transmitting the program. Note that the invention can be realized also in a form of a data signal embedded in carrier waves, into which the program is embodied by electronic transmission.

### Overview

An information management apparatus according to an aspect 1 of the invention is an information management apparatus 2 provided with a display unit 20, which includes a reception unit (data reception unit 11) that receives measurement information in at least two different formats, which is transmitted from a plurality of measurement devices which have measured a subject, and a processing unit (data generation unit 13, data notification unit 14) that generates, from the received measurement information, notification data by using a common parameter in the plurality of measurement devices and notifies the display unit of the notification data.

According to the aforementioned configuration, the information management apparatus generates, from the measurement information in at least two different formats received from the measurement devices, the notification data formed of the parameter common in each of the measurement devices, and notifies the display unit for display. Accordingly, it is not required to make a design nor a configuration of a common notification format complex.

In the information management apparatus according to an aspect 2 of the invention, the reception unit (data reception unit 11) may receive unique information of a measurement device, and the processing unit (data generation unit 13) may generate notification data which includes, as the common parameter, a device unique information identifier for identifying the unique information in the aspect 1.

According to the aforementioned configuration, it is possible to notify the display unit of the device unique information identifier without changing the common notification format. Further, by using the device unique information identifier stored in the notification data, the display unit is able to acquire unique information of the measurement device.

The information management apparatus according to an aspect 3 of the invention may include a unique information recording unit (data accumulation unit 40) that records at least the device unique information identifier and the unique information, in which the display unit 20 may acquire, from the unique information recording unit, and display the unique information linked to the device unique information identifier stored in the notification data which has been notified, in the aspect 2.

According to the aforementioned configuration, it is possible to inform a user of the unique information acquired by the information management apparatus.

In the information management apparatus according to an aspect 4 of the invention, the reception unit (data reception unit 11) may receive subject information of the subject, and the processing unit (data generation unit 13) may generate notification data which includes, as the common parameter, a subject information identifier for identifying the subject information, in the aspects 1 to 3.

According to the aforementioned configuration, it is possible to notify the display unit of the subject information identifier without changing the common notification format. Further, by using the subject information identifier stored in the notification data, the display unit is able to acquire the subject information.

The information management apparatus according to an aspect 5 of the invention may include a subject information recording unit (data accumulation unit 40) that records the subject information identifier and the subject information, in which the display unit 20 may acquire, from the subject information recording unit, and display the subject information linked to the subject information identifier stored in the notification data which has been notified, in the aspect 4.

According to the aforementioned configuration, it is possible to notify the user of the subject information acquired by the information management apparatus.

In the information management apparatus according to an aspect 6 of the invention, the processing unit (data generation unit 13) may generate, from the measurement information for a plurality of times which is received from a same measurement device, notification data in a manner of repeating parameters of the measurement information for one time, in the aspects 1 to 5.

According to the aforementioned configuration, even when the measurement information for a plurality of times is received, the information management apparatus is able to display a measurement result normally.

In the information management apparatus according to an aspect 7 of the invention, the processing unit (data generation unit 13) may generate notification data in a format including a character string representing a type of a measurement result and a value of the measurement result, in the aspects 1 to 6.

According to the aforementioned configuration, the information management apparatus is able to inform the user of the type and the value of the measurement result.

In the information management apparatus according to an aspect 8 of the invention, the measurement information may be biological information of the subject in the aspects 1 to 7.

According to the aforementioned configuration, the information management apparatus is able to display the biological information of the subject.

The information management apparatus according to an aspect 9 of the invention may include a plurality of display units 20, in which the processing unit (data notification unit 14) may notify all the plurality of display units 20 of the notification data, in the aspects 1 to 8.

According to the aforementioned configuration, even when there are a plurality of display units, the measurement result is able to be displayed by using all the display units.

The information management apparatus according to an aspect 10 of the invention may include a plurality of display units 20, in which the processing unit (data notification unit 14) may notify only a specific display unit 20 among the plurality of display units 20 of the notification data, in the aspects 1 to 8.

According to the aforementioned configuration, even when there are a plurality of display units, the measurement result is displayed only on the display unit on which display is desired to be performed, thus making it possible to control a flow of the notification data.

An information management apparatus according to an aspect 11 of the invention is an information management apparatus 2 that communicates with an information accumulation apparatus, which may include a reception unit (data reception unit 11) that receives measurement information in at least two different formats, which is transmitted from a plurality of measurement results which have measured a subject, and a processing unit (data generation unit 13, data notification unit 14) that generates, from the received measurement result, notification data by using a common parameter in the plurality of measurement devices and notifies the information accumulation apparatus of the notification data.

According to the aforementioned configuration, it is not required to make a design nor a configuration of the common notification format complex.

The information management apparatus according to each aspect of the invention may be realized by a computer, and in such a case, a control program of the information management apparatus, which realizes the information management apparatus in the computer by causing the computer to operate as each means included in the information management apparatus, and a computer readable recording medium having it recorded therein are also incorporated in a range of the invention.

The invention is not limited to each of the embodiments described above, and can be modified variously within the scope defined by the claims, and embodiments obtained by appropriately combining technical means disclosed in different embodiments are also included in the technical scope of the invention. Further, by combining the technical means disclosed in each of the embodiments, a new technical feature may be formed.

### INDUSTRIAL APPLICABILITY

The invention is able to be used for an information terminal, such as a mobile phone or a smart phone, which receives measurement information form measurement devices which have measured biological information and displays the measurement information.

### REFERENCE SIGNS LIST

1 information management system
2 information management apparatus
3 network
4 device
8 display unit distribution apparatus
9 information accumulation apparatus
10 processing unit
11 data reception unit (reception unit)
12 data analysis unit
13 data generation unit (processing unit)
14 data notification unit (processing unit)
20 display unit
21 display data extraction unit
22 accumulated data acquisition unit
30 display panel
40 data accumulation unit (unique information recording unit, subject information recording unit)
50 user input unit
60 display unit acquisition unit
70 communication unit

1. An information management apparatus provided with a display unit, comprising:
a reception unit that receives measurement information in at least two different formats, which is transmitted from a plurality of measurement devices which have measured a subject; and
a processing unit that generates, from the received measurement information, notification data by using a parameter common in the plurality of measurement devices and notifies the display unit of the notification data.

2. The information management apparatus according to claim 1, wherein
the reception unit receives unique information of the plurality of measurement devices, and
the processing unit generates notification data which includes, as the common parameter, a device unique information identifier for identifying the unique information.

3. The information management apparatus according to claim 2, further comprising:
a unique information recording unit that records at least the device unique information identifier and the unique information, wherein
the display unit acquires, from the unique information recording unit, and displays the unique information linked to the device unique information identifier stored in the notification data which has been notified.

4. The information management apparatus according to claim 1, wherein
the reception unit receives subject information of the subject, and
the processing unit generates notification data which includes, as the common parameter, a subject information identifier for identifying the subject information.

5. The information management apparatus according to claim 4, further comprising:
a subject information recording unit that records the subject information identifier and the subject information, wherein
the display unit acquires, from the subject information recording unit, and displays the subject information linked to the subject information identifier stored in the notification data which has been notified.

6. The information management apparatus according to claim 1, wherein the processing unit generates, from the measurement information for a plurality of times which is received from a same measurement device, notification data in a manner of repeating parameters of the measurement information for one time.

7. The information management apparatus according to claim 1, wherein the processing unit generates notification data in a format including a character string representing a type of the measurement information and a value of the measurement information.

8. The information management apparatus according to claim 1, wherein the measurement information is biological information of the subject.

9. The information management apparatus according to claim 1, further comprising:
a plurality of display units, wherein
the processing unit notifies all the plurality of display units of the notification data.

10. The information management apparatus according to claim 1, further comprising:
a plurality of display units, wherein
the processing unit notifies only a specific display unit among the plurality of display units of the notification data.

11. An information management apparatus that communicates with an information accumulation apparatus, comprising:
a reception unit that receives measurement information in at least two different formats, which is transmitted from a plurality of measurement devices which have measured a subject, and
a processing unit that generates, from the received measurement information, notification data by using a common parameter in the plurality of measurement devices and notifies the information accumulation apparatus of the notification data.
